(19) United States
(12) Patent Application Publication
(10) Pub. No.: US 2021/0128871 A1
(43) Pub. Date: May 6, 2021

(54) GUIDEWIRE ASSEMBLY DETECTABLE BY MEDICAL-IMAGING SENSOR

(71) Applicant: Baylis Medical Company Inc., Montreal (CA)

(72) Inventors: Krishnanand Balasundaram, Pickering (CA); John Paul Urbanski, Toronto (CA)

(21) Appl. No.: 17/084,084

(22) Filed: Oct. 29, 2020

Related U.S. Application Data
(60) Provisional application No. 62/928,214, filed on Oct. 30, 2019.

(51) Int. Cl. A61M 25/01; A61M 25/09; A61B 18/14; A61B 90/00

(52) U.S. Cl. CPC A61M 25/0105; A61M 25/09; A61B 2018/00625; A61B 90/37; A61B 18/1492

(57) ABSTRACT

A flexible guidewire assembly has an energy-emitting device configured to be selectively activated in response to receiving a first activation signal transmittable from a control assembly. This is done in such a way that the energy-emitting device, once activated, emits a first amount of electro-magnetic energy. The first amount of electro-magnetic energy has a first strength that is (A) sufficient for detection by a medical-imaging sensor positioned in the confined space defined by the living body, and (B) insufficient for vaporization of any tissue of the living body.

### TECHNICAL FIELD

This document relates to the technical field of (and is not limited to) a guidewire assembly (and method therefor); more specifically, this document relates to the technical field of (and is not limited to) a guidewire assembly configured to be detected by a medical-imaging sensor (and method therefor).

### BACKGROUND

Known medical devices are configured to facilitate a medical procedure, and help healthcare providers diagnose and/or treat medical conditions of sick patients.

### SUMMARY

It will be appreciated that there exists a need to mitigate (at least in part) at least one problem associated with the existing guidewires (also called the existing technology). After much study of, and experimentation with, the existing guidewires, an understanding (at least in part) of the problem and its solution have been identified (at least in part) and are articulated (at least in part) as follows:

The use of minimally invasive devices to gain access to and perform procedures on the various surfaces of the heart may be required for the diagnosis and/or treatment of various cardiac complications. In order to perform such procedures, a cardiologist or electrophysiologist may need to gain access to the desired surface (be it left atrium access or the epicardial surface access). This typically may require a catheter to create a puncture in either the Fossa Ovalis (FO) in the case of left atrium access or the pericardial sac in the case of epicardial surface access. In order to gain access, a physician may be able to visualize the desirable site that he or she wishes to puncture.

The traditional imaging modality used to visualize the minimally invasive catheters has been fluoroscopy. However, due to the radiation effects of fluoroscopy, the industry has begun to switch to other imaging modalities in order to visualize such catheters. One such modality is echocardiography, more specifically, Intracardiac Echocardiography (ICE) and Transesophagael Echocardiography (TEE). These modalities may rely on using ultrasound waves to reflect off their surroundings or mediums (such as, the surrounding tissue of the patient, other medical devices, a catheter, etc.) to create an image of its surrounding. Even with catheters having echogenic properties, it is often difficult to visualize the tip of the puncture device in the environment, due to common interferences observed in said environment (tissue, blood, other catheters such as dilators, etc.). If the physician has difficulty visualizing the tip of the puncture device, then there is less confidence in gaining access and more potential for complications.

Given that visualization of the tip of the puncture device (during a medical procedure) may be important prior to puncturing the target site (located on the tissue of the patient), it may be helpful to arrange the tip (of the puncture device) to be highly echogenic on demand to be easily visualized on ICE (Intracardiac Echocardiography) or TEE (Transesophageal Echocardiogram). Echogenicity is the ability of tissue to reflect a signal (such as an acoustic signal, etc.). This may not always be possible for several reasons. A first reason may be that the ICE or TEE may have to visualize the puncture device through tissue (i.e. looking for the epicardial puncture site through the ventricles (of the patient) in the case of ICE or through the esophagus in the case of TEE). A second reason may be that a dilator device and/or an introducer device may be typically utilized to align the puncture device onto the puncture site. These dilators and/or introducers may create interference (from their own echogenic materials), which make it more difficult to visualize the puncture device.

What may be beneficial is a way to identify a puncture device on the echocardiography image (to be generated and/or displayed by a medical imaging system). The puncture device may create an interference to (on) the echocardiography image, with the source of the interference being, for instance, provided by the electromagnetic energy (such as, radio frequency waves, etc., and any equivalent thereof) emitted from the tip of the puncture device. Enabling the viewing of the electromagnetic energy (emitted from the puncture device by the cardiologist and/or electrophysiologist) on the echocardiography image may make the puncture device more visible (for the cardiologist and/or electrophysiologist). In this manner, the medical professional may be able to proceed with medical treatment with less time, frustration and/or effort.

To mitigate, at least in part, at least one problem associated with the existing technology, there is provided (in accordance with a major aspect) an apparatus.

The apparatus includes a flexible guidewire assembly that is insertable into, and is movable along, a confined space defined by a living body. The flexible guidewire assembly has an energy-emitting device. The energy-emitting device is configured to be selectively activated in response to the energy-emitting device, in use, receiving a first activation signal being transmittable from a control assembly (in such a way that the energy-emitting device, once activated, emits a first amount of electro-magnetic energy under a first case while the energy-emitting device of the flexible guidewire assembly is movable along the confined space defined by the living body). Under the first case, the first amount of electro-magnetic energy has a first strength that is: (A) sufficient (strong enough) for detection by a medical-imaging sensor positioned in the confined space defined by the living body; and (B) insufficient (not strong enough) for vaporization of any tissue of the living body.

To mitigate, at least in part, at least one problem associated with the existing technology, there is provided (in accordance with a major aspect) an apparatus.

The apparatus includes a control assembly configured to transmit a first activation signal to an energy-emitting device of a flexible guidewire assembly being configured to be selectively activated in response to the energy-emitting device, in use, receiving the first activation signal in such a way that the energy-emitting device, once activated, emits a first amount of electro-magnetic energy under a first case while the energy-emitting device of the flexible guidewire assembly is movable along the confined space defined by the living body, in which the flexible guidewire assembly is insertable into, and movable along, a confined space defined by a living body. Under the first case, the first amount of electro-magnetic energy has a first strength that is: (A) sufficient (strong enough) for detection by a medical-imaging sensor positioned in the confined space defined by the living body; and (B) insufficient (not strong enough) for vaporization of any tissue of the living body.

To mitigate, at least in part, at least one problem associated with the existing technology, there is provided (in accordance with a major aspect) a computer program product for a control assembly, including executable programmed code for directing the control assembly to transmit a first activation signal to an energy-emitting device of a flexible guidewire assembly being configured to be selectively activated in response to the energy-emitting device, in use, receiving the first activation signal in such a way that the energy-emitting device, once activated, emits a first amount of electro-magnetic energy under a first case while the energy-emitting device of the flexible guidewire assembly is movable along the confined space defined by the living body, in which the flexible guidewire assembly is insertable into, and being movable along, a confined space defined by a living body. Under the first case, the first amount of electro-magnetic energy has a first strength that is: (A) sufficient (strong enough) for detection by a medical-imaging sensor positioned in the confined space defined by the living body; and (B) insufficient (not strong enough) for vaporization of any tissue of the living body.

To mitigate, at least in part, at least one problem associated with the existing technology, there is provided (in accordance with a major aspect) a method of utilizing a flexible guidewire assembly including an energy-emitting device, the method including (A) identifying and utilizing a vein positioned in the right atrium (of the patient); and (B) introducing the medical-imaging sensor into the interior of the vein so that the medical-imaging sensor enters the right atrium; and (C) establishing electrical communication between the medical-imaging sensor and a medical-imaging system; and (D) energizing the medical-imaging sensor so that the medical-imaging sensor transmits a sensor signal to the tissue of the patient, in which the tissues of the patient reflect back at least some of the sensor signal toward the medical-imaging sensor, and the medical-imaging sensor transmits the reflected sensor signal back to the medical-imaging system; and (E) introducing a dilator assembly and the energy-emitting device into the interior of the vein so that the dilator assembly and the energy-emitting device enter the right atrium; and (F) moving the dilator assembly toward the tissue wall so that the dilator assembly makes intimate contact with the tissue wall; and (G) establishing electrical communication between a control assembly and the energy-emitting device of the flexible guidewire assembly. In accordance with an option, the method further includes activating the energy-emitting device to emit a first amount of electro-magnetic energy once the energy-emitting device is positioned in a spaced-apart relationship with a tissue wall so that the position of the energy-emitting device is visualized via synergistic cooperation between (of) the medical-imaging sensor and the medical-imaging system without causing any vaporization of the tissue of the patient. In accordance with an option, the method further includes activating the energy-emitting device to emit a second amount of electro-magnetic energy once the energy-emitting device is positioned proximate to the tissue wall so that the energy-emitting device forms a passageway through the tissue wall by vaporizing a portion of the tissue wall.

Other aspects are identified in the claims. Other aspects and features of the non-limiting embodiments may now become apparent to those skilled in the art upon review of the following detailed description of the non-limiting embodiments with the accompanying drawings. This Summary is provided to introduce concepts in simplified form that are further described below in the Detailed Description. This Summary is not intended to identify potentially key features or possible essential features of the disclosed subject matter, and is not intended to describe each disclosed embodiment or every implementation of the disclosed subject matter. Many other novel advantages, features, and relationships will become apparent as this description proceeds. The figures and the description that follow more particularly exemplify illustrative embodiments.

### BRIEF DESCRIPTION OF THE DRAWINGS

The non-limiting embodiments may be more fully appreciated by reference to the following detailed description of the non-limiting embodiments when taken in conjunction with the accompanying drawings, in which:

FIG. 1 (SHEET 1 of 8 SHEETS) depicts a top schematic view of an embodiment of a flexible guidewire assembly having an energy-emitting device (in which the energy-emitting device is not activated, and is positioned in a spaced-apart relationship with a tissue wall); and

FIG. 2 (SHEET 1 of 8 SHEETS) depicts a top medical-image view of an embodiment of the flexible guidewire assembly of FIG. 1 (in which the energy-emitting device is not activated, and is positioned in a spaced-apart relationship with a tissue wall); and

FIG. 3 (SHEET 2 of 8 SHEETS) depicts a top schematic view of the embodiment of the flexible guidewire assembly and the energy-emitting device of FIG. 1 (in which the energy-emitting device is activated and emits a first amount of electro-magnetic energy, and is positioned in a spaced-apart relationship with a tissue wall); and

FIG. 4 (SHEET 2 of 8 SHEETS) depicts a top medical-image view of the embodiment of the flexible guidewire assembly and the energy-emitting device of FIG. 3 (in which the energy-emitting device is activated and emits a first amount of electro-magnetic energy, and is positioned in a spaced-apart relationship with a tissue wall); and

FIG. 5 (SHEET 3 of 8 SHEETS) depicts a top schematic view of the embodiment of the flexible guidewire assembly and the energy-emitting device of FIG. 1 (in which the energy-emitting device is activated and emits a first amount of electro-magnetic energy, and is positioned proximate to (in intimate contact with) a tissue wall); and

FIG. 6 (SHEET 3 of 8 SHEETS) depicts a top medical-image view of the embodiment of the flexible guidewire assembly and the energy-emitting device of FIG. 5 (in which the energy-emitting device is activated and emits a first amount of electro-magnetic energy, and is positioned proximate to (in intimate contact with) a tissue wall); and

FIG. 7 (SHEET 4 of 8 SHEETS) depicts a top schematic view of the embodiment of the flexible guidewire assembly and the energy-emitting device of FIG. 1 (in which the energy-emitting device is deactivated, and is positioned proximate to (in intimate contact with) a tissue wall); and

FIG. 8 (SHEET 4 of 8 SHEETS) depicts a top medical-image view of the embodiment of the flexible guidewire assembly and the energy-emitting device of FIG. 7 (in which the energy-emitting device is deactivated, and is positioned proximate to (in intimate contact with) a tissue wall); and

FIG. 9 (SHEET 5 of 8 SHEETS) depicts a top schematic view of the embodiment of the flexible guidewire assembly and the energy-emitting device of FIG. 1 (in which the energy-emitting device is activated to emit a second amount of electro-magnetic energy while positioned proximate to (in intimate contact with) a tissue wall); and

FIG. 10 (SHEET 5 of 8 SHEETS) depicts a top medical-image view of the embodiment of the flexible guidewire assembly and the energy-emitting device of FIG. 9 (in which the energy-emitting device is activated to emit a second amount of electro-magnetic energy while positioned proximate to (in intimate contact with) a tissue wall); and

FIG. 11 (SHEET 6 of 8 SHEETS) depicts a top schematic view of the embodiment of the flexible guidewire assembly and the energy-emitting device of FIG. 1 (in which the energy-emitting device is activated to emit a second amount of electro-magnetic energy while positioned in a spaced-apart relationship with a tissue wall); and

FIG. 12 (SHEET 6 of 8 SHEETS) depicts a top medical-image view of the embodiment of the flexible guidewire assembly and the energy-emitting device of FIG. 11 (in which the energy-emitting device is activated to emit a second amount of electro-magnetic energy while positioned in a spaced-apart relationship with a tissue wall); and

FIG. 13 (SHEET 7 of 8 SHEETS) depicts a top schematic view of the embodiment of the flexible guidewire assembly and the energy-emitting device of FIG. 1 (in which the energy-emitting device is deactivated while positioned in a spaced-apart relationship with a tissue wall); and

FIG. 14 (SHEET 7 of 8 SHEETS) depicts a top medical-image view of the embodiment of the flexible guidewire assembly and the energy-emitting device of FIG. 13 (in which the energy-emitting device is deactivated while positioned in a spaced-apart relationship with a tissue wall); and

FIG. 15 and FIG. 16 (SHEET 8 of 8 SHEETS) depict top schematic views of the embodiments of the flexible guidewire assembly and the energy-emitting device of FIG. 1 (which depict cases to be, preferably, avoided).

The drawings are not necessarily to scale and may be illustrated by phantom lines, diagrammatic representations and fragmentary views. In certain instances, details unnecessary for an understanding of the embodiments (and/or details that render other details difficult to perceive) may have been omitted. Corresponding reference characters indicate corresponding components throughout the several figures of the drawings. Elements in the several figures are illustrated for simplicity and clarity and have not been drawn to scale. The dimensions of some of the elements in the figures may be emphasized relative to other elements for facilitating an understanding of the various disclosed embodiments. In addition, common, and well-understood, elements that are useful in commercially feasible embodiments are often not depicted to provide a less obstructed view of the embodiments of the present disclosure.

### LISTING OF REFERENCE NUMERALS USED IN THE DRAWINGS

flexible guidewire assembly 102

dilator assembly 103

energy-emitting device 104

vein 106

terminal assembly 107

first case 201

first amount of electro-magnetic energy 202

second case 204

second amount of electro-magnetic energy 206

medical-imaging sensor 700

sensor field-of-view 701 (FOV)

medical-imaging system 702

medical image 704

control assembly 800

first activation signal 802

first deactivation signal 804

second activation signal 806

second deactivation signal 808

confined space 900

living body 902

heart 903

tissue wall 904

atrial septum 905

passageway 906

right atrium 908

left atrium 910

### DETAILED DESCRIPTION OF THE NON-LIMITING EMBODIMENT(S)

The following detailed description is merely exemplary and is not intended to limit the described embodiments or the application and uses of the described embodiments. As used, the word “exemplary” or “illustrative” means “serving as an example, instance, or illustration.” Any implementation described as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure. The scope of the claim is defined by the claims (in which the claims may be amended during patent examination after the filing of this application). For the description, the terms “upper,” “lower,” “left,” “rear,” “right,” “front,” “vertical,” “horizontal,” and derivatives thereof shall relate to the examples as oriented in the drawings. There is no intention to be bound by any expressed or implied theory in the preceding Technical Field, Background, Summary or the following detailed description. It is also to be understood that the devices and processes illustrated in the attached drawings, and described in the following specification, are exemplary embodiments (examples), aspects and/or concepts defined in the appended claims. Hence, dimensions and other physical characteristics relating to the embodiments disclosed are not to be considered as limiting, unless the claims expressly state otherwise. It is understood that the phrase “at least one” is equivalent to “a”. The aspects (examples, alterations, modifications, options, variations, embodiments and any equivalent thereof) are described regarding the drawings. It should be understood that the invention is limited to the subject matter provided by the claims, and that the invention is not limited to the particular aspects depicted and described. It will be appreciated that the scope of the meaning of a device configured to be coupled to an item (that is, to be connected to, to interact with the item, etc.) is to be interpreted as the device being configured to be coupled to the item, either directly or indirectly. Therefore, “configured to” may include the meaning “either directly or indirectly” unless specifically stated otherwise.

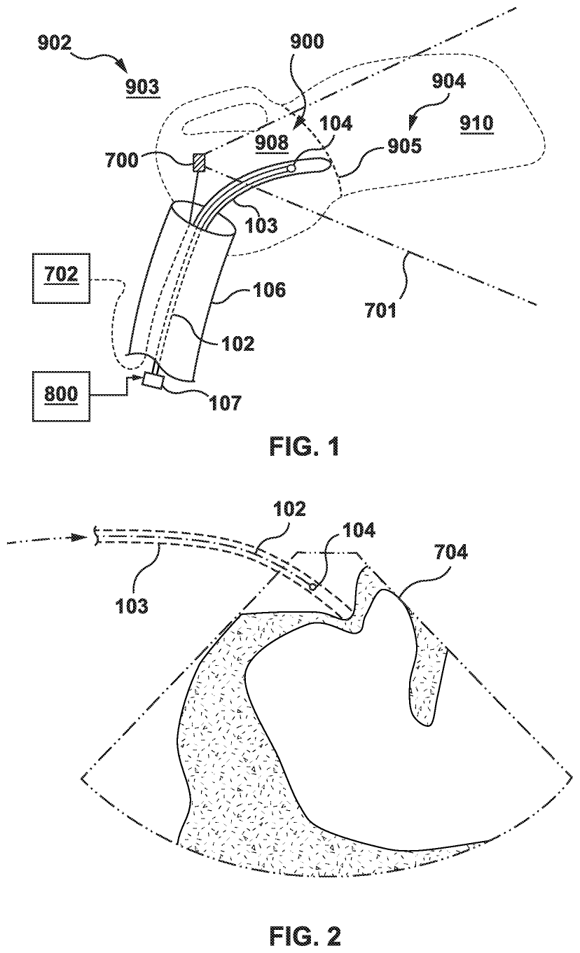

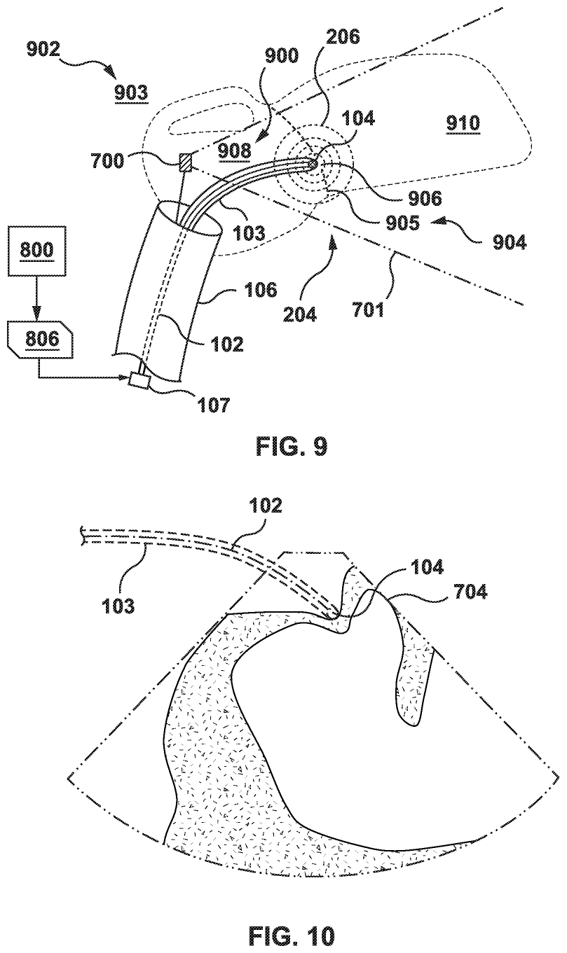

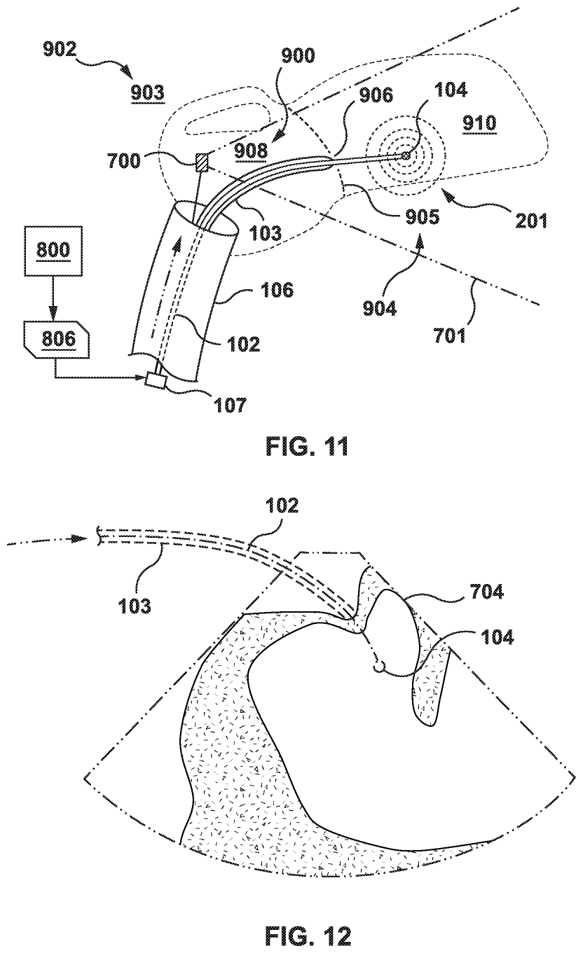

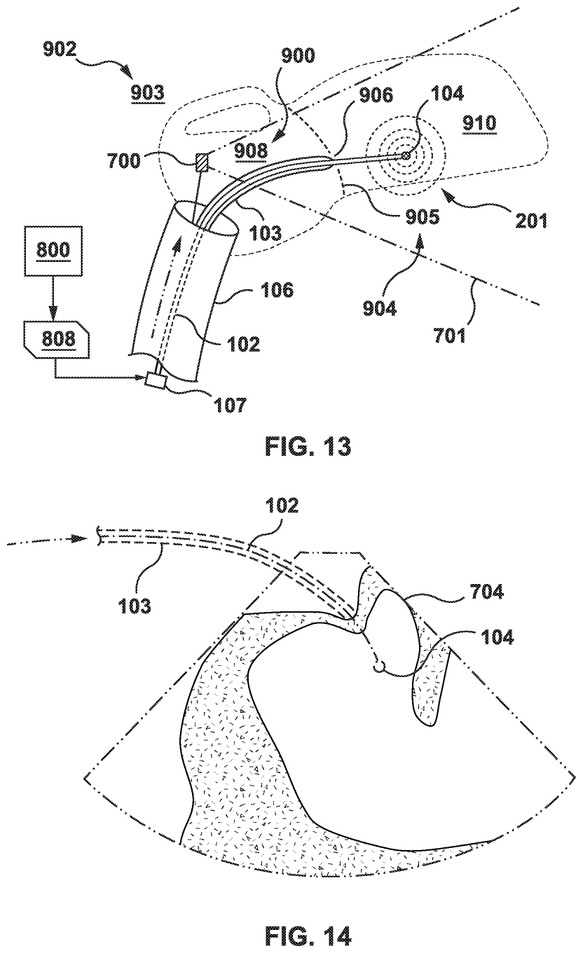

FIG. 1 depicts a top schematic view of an embodiment of a flexible guidewire assembly 102 having an energy-emitting device 104. FIG. 2 depicts a top medical-image view of an embodiment of the flexible guidewire assembly 102 of FIG. 1. In accordance with FIG. 1 and FIG. 2, the energy-emitting device 104 is not activated (not energized), and is positioned in a spaced-apart relationship with a tissue wall 904. The flexible guidewire assembly 102 is placed in position, ultimately, for the formation of a passageway 906 extending through the tissue wall 904 (the passageway 906 is depicted in FIG. 9, FIG. 11 and FIG. 13) It will be appreciated that FIG. 1 depicts a condition prior to the formation of the passageway 906.

Referring to the embodiment as depicted in FIG. 1, a flexible guidewire assembly 102 is insertable into, and is movable along, a confined space 900 (such as the interior of the heart 903). The confined space 900 is defined by a living body 902 (a patient, etc.). The flexible guidewire assembly 102 has an energy-emitting device 104. The flexible guidewire assembly 102 is (preferably) impermeable by a bodily fluid of the patient. The flexible guidewire assembly 102 (preferably) has a circular cross-sectional section or profile; it will be appreciated that other profile shapes may be utilized. Preferably, the energy-emitting device 104 (also called a controllable electrode, etc.) is fixedly positioned on (mounted to) a distal tip portion of the flexible guidewire assembly 102. Preferably, the energy-emitting device 104 is configured to emit electromagnetic energy. Electromagnetic energy includes (and is not limited to) any form of energy that may be reflected and/or emitted from objects through energy waves (such as electrical waves and/or magnetic waves, and any equivalent thereof) traveling through space (in which the energy waves are detectable by a sensor configured to detect such waves). For instance, electromagnetic energy may include (and is not limited to) gamma rays, X-rays, ultraviolet radiation, visible light, microwaves, radio waves and infrared radiation, ultrasound waves, and any equivalent thereof. In accordance with an embodiment, the energy-emitting device 104 is configured to emit ultrasound waves. The energy-emitting device 104 includes an ultrasound transceiver (known and not depicted) configured to emit ultrasound waves. A conductive tip or a portion of the energy-emitting device 104 may include a material, a surface area and/or an outer diameter that may be varied or adapted to facilitate transmission of electromagnetic energy toward the tissue wall 904 (once the energy-emitting device 104 is positioned and activated as depicted in the embodiment of FIG. 9). The flexible guidewire assembly 102 may include an insulation component; the material and thickness may be varied to better insulate an electrical conductor positioned within the interior of the flexible guidewire assembly 102 (the electrical conductor is utilized to activate the energy-emitting device 104). Reference is made to the following publication for consideration in the selection of suitable materials: Plastics in Medical Devices: Properties, Requirements, and Applications; 2nd Edition; author: Vinny R. Sastri; hardcover ISBN: 9781455732012; published: 21 Nov. 2013; publisher: Amsterdam [Pays-Bas]: Elsevier/William Andrew, [2014].

Referring to the embodiment as depicted in FIG. 1, the flexible guidewire assembly 102 may include any type of a flexible material that is compatible for medical usage. The energy-emitting device 104 is configured to selectively emit electromagnetic energy (in response to the energy-emitting device 104 receiving a command signal (an ON signal or an OFF signal, etc.) from a control assembly 800. The energy-emitting device 104 is, preferably, configured to selectively emit electromagnetic energy (such as, and not limited to, radio frequency energy, ultrasound waves, etc.). For instance the energy-emitting device 104 may include an ultrasound sensor (device), etc.

Referring to the embodiment as depicted in FIG. 1, the living body 902 (also called the patient) includes, preferably, a heart 903, or other organ, etc. The confined space 900 includes, for instance, the right atrium 908 and the left atrium 910 of the heart 903. A tissue wall 904 (of the heart 903) includes the atrial septum 905 that separates the right atrium 908 and the left atrium 910 of the heart 903.

Referring to the embodiment as depicted in FIG. 1, the flexible guidewire assembly 102 is (preferably) configured to be receivable within an interior elongated passageway of (defined by) a dilator assembly 103. The dilator assembly 103 has a dilation device mounted to the front end of the dilator assembly 103. The flexible guidewire assembly 102 is configured to be movable along an elongated length of the dilator assembly 103 (and along the interior passageway of the dilator assembly 103). The dilator assembly 103 is insertable into, and is movable along, the confined space 900 (such as the interior of the heart 903), and the confined space 900 is defined by the living body 902 (a patient, etc.). The dilator assembly 103 (preferably) has a circular cross-sectional section or profile (it will be appreciated that other profile shapes may be utilized). The flexible guidewire assembly 102 is movable such that the energy-emitting device 104 may be movable toward the tissue wall 904 (preferably, along and within the dilator assembly 103), as depicted in FIG. 1). It will be appreciated that once the energy-emitting device 104 is activated (as depicted in FIG. 9) for the purpose of forming an initial pilot hole (the passageway 906) through the tissue wall 904, the dilator assembly 103 may be utilized to further ream out (mechanically ream) the passageway 906 thereby further enlarging the size of the passageway 906.

Referring to the embodiment as depicted in FIG. 1, the dilator assembly 103 is configured to be receivable along a vein 106. For instance, the vein 106 brings (conveys) blood into the right side of the heart 903 of the patient (more specifically, into the inferior-vena cava of the heart 903, as depicted in FIG. 1). The vein 106 is positioned within the confined space 900, such as the right atrium 908 of the heart 903, etc.). The dilator assembly 103 and the flexible guidewire assembly 102 are inserted into, and moved along, the vein 106.. It may be appreciated that an equivalent structure of the vein 106 may include a medical-tube assembly, and any equivalent thereof (whether being and/or having an artificial material and/or a biological material, etc.). The vein 106 represents a tube (generally). The preferred embodiment utilizes the vein (as depicted) as a preferred arrangement (or structure) for cooperating with the dilator assembly 103.

Referring to the embodiment as depicted in FIG. 1, the flexible guidewire assembly 102 includes a terminal assembly 107 positioned at a proximal end of the flexible guidewire assembly 102. The terminal assembly 107 is configured to be electrically connectable to the energy-emitting device 104; preferably, an electrical wire (known and not depicted) is positioned within and along a length of the flexible guidewire assembly 102 for electrically connecting the terminal assembly 107 to the energy-emitting device 104. The control assembly 800 is configured to be electrically connectable to the energy-emitting device 104 via the terminal assembly 107. The control assembly 800 is configured to selectively provide (convey) electromagnetic energy to the energy-emitting device 104 (once the control assembly 800 is electrically connected to the energy-emitting device 104 via the terminal assembly 107, etc.). The control assembly 800 may include, for instance, a processor, a memory assembly, and executable programmed code (stored in the memory assembly), and is configured to direct the processor to execute controller operations for the control of the operations of the components of the control assembly 800, etc. The control assembly 800 may include, for instance, a radio frequency generator. The components of the control assembly 800 may be modified to create (generate or provide) an optimal electromagnetic signal (having a frequency and an amplitude, etc.) to be viewed ( ) by a medical-imaging sensor 700 (which is positioned proximate to the energy-emitting device 104). The control assembly 800 is configured to (preferably) produce radio frequency pulses that may be detected by the medical-imaging sensor 700 (via the energy emanating from the energy-emitting device 104). The control assembly 800 may include, for instance, the MODEL RFP-100A RF PUNCTURE GENERATOR system manufactured or supplied by BAYLIS MEDICAL headquartered in Ontario, Canada. The control assembly 800 is known and not further described herein. In accordance with an option, the energy-emitting device 104 may include an echogenic material thereby making the energy-emitting device 104 more visible on echocardiography, etc. In accordance with an option, the medical-imaging sensor 700 is mounted to the flexible guidewire assembly 102, and is positioned proximate to the energy-emitting device 104.

Referring back to the embodiment as depicted in FIG. 1, the terminal assembly 107 (also called a proximal connector) is configured to connect the energy-emitting device 104 to the control assembly 800 (such as, a radio frequency generator, etc.). An electrically conductive wire (also called a conductive mandrel) connects the terminal assembly 107 to the energy-emitting device 104 (in which the electrical wire (not depicted) is embedded along a longitudinal length of the flexible guidewire assembly 102). The electrically conductive wire has, preferably, a low impedance to prevent dissipation of electromagnetic energy. The flexible guidewire assembly 102 includes suitable insulation configured to prevent degradation of electromagnetic energy along the shaft or a longitudinal length of the flexible guidewire assembly 102. The electrically conductive wire (also called a mandrel) connects the energy-emitting device 104 to the control assembly 800 (via the terminal assembly 107). The material and outer diameter may be changed to reduce the impedance of the wire, etc.

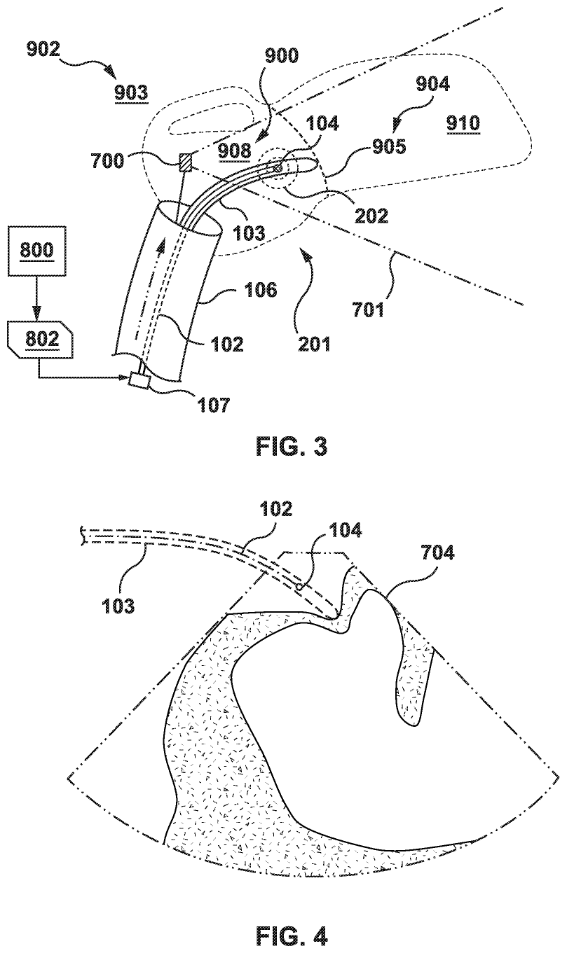

Referring to the embodiment as depicted in FIG. 1, a medical-imaging sensor 700 is configured to be positioned in the confined space 900 defined by the living body 902. The medical-imaging sensor 700 is positioned proximate to the tissue wall 904. The medical-imaging sensor 700 is configured to be positioned at a distal end of the vein 106. The medical-imaging sensor 700 is configured to transmit and receive a sensor signal (by way of transmitting a signal and detecting reflections of the signal from the surrounding tissues of the patient). The medical-imaging sensor 700 is configured to transmit the sensor signal toward the interior tissues (such as the tissue wall 904, etc.) of the patient, and a portion of the sensor signal is reflected from the interior tissues of the patient back to the medical-imaging sensor 700. For the case where the energy-emitting device 104 is deactivated (as depicted in FIG. 1), the medical-imaging sensor 700 is not able to detect the presence of the energy-emitting device 104. For the case where the energy-emitting device 104 is activated (as depicted in FIG. 3), the medical-imaging sensor 700 is able to detect the presence of the energy-emitting device 104. The emitted energy signal (generated by the energy-emitting device 104) is received by (and is sensed by) the medical-imaging sensor 700. The medical-imaging sensor 700 is configured to transmit information about the reflected sensor signal to a medical-imaging sensor 700. An embodiment of the medical-imaging sensor 700 includes an intracardiac echocardiography sensor (an ICE sensor). The medical-imaging sensor 700 is known and not further described herein. The medical-imaging sensor 700 has a sensor field-of-view 701 (FOV) once the medical-imaging sensor 700 is activated accordingly. The sensor field-of-view 701 is utilized, by the medical-imaging system 702, to view a portion of the right atrium 908 and a portion of the left atrium 910.

Referring to the embodiment as depicted in FIG. 1, a medical-imaging system 702 is configured to be electrically connected to the medical-imaging sensor 700. The medical-imaging system 702 is configured to generate a medical image 704 that is formed based on the sensor data (information) provided by the medical-imaging sensor 700, and received by the medical-imaging system 702. The medical-imaging system 702 may include, for instance, a system of echocardiography, such as transthoracic echocardiogram (TTE), transesophageal echocardiogram (TEE), Intracardiac echocardiography (ICE), and/or Intravascular ultrasound (IVUS), and any equivalent thereof. The medical-imaging system 702 is known and not further described herein.

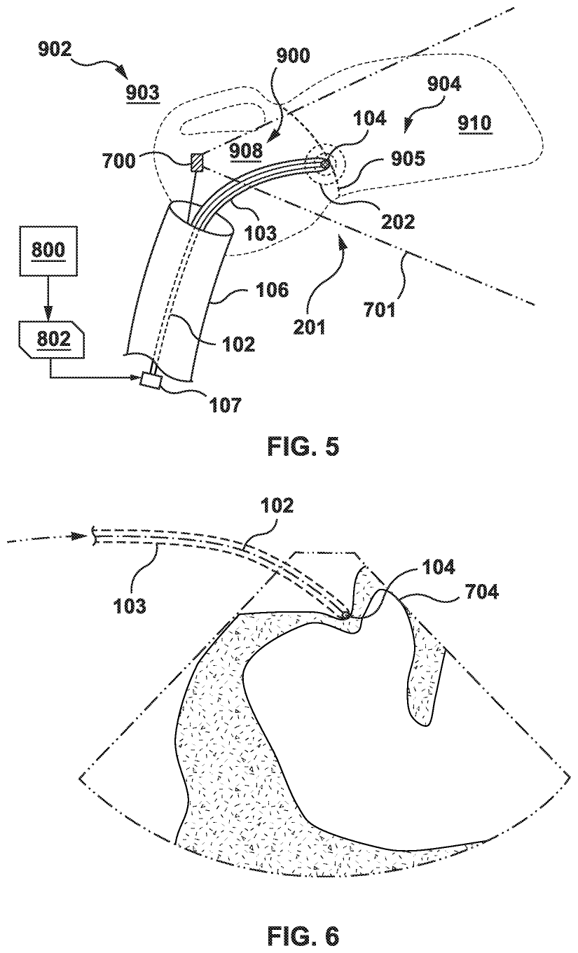

Referring to the embodiment as depicted in FIG. 1, the medical-imaging sensor 700 is configured to be in signal communication with a medical-imaging system 702 (preferably while the medical-imaging sensor 700, in use, is positioned in the confined space 900 defined by the living body 902). The medical-imaging system 702 is configured to receive a sensor signal transmitted by the medical-imaging sensor 700 (preferably while the medical-imaging sensor 700, in use, is positioned in the confined space 900 defined by the living body 902). The medical-imaging system 702 is configured to display, to a user (such as a doctor, not depicted) via a display device (known and not depicted), a medical image 704 (as depicted in FIG. 2) depicting a first relative position of an emission of the first amount of electro-magnetic energy 202 in response to the medical-imaging system 702, in use, receiving the sensor signal transmitted by the medical-imaging sensor 700. This is, preferably, done (A) while the medical-imaging sensor 700, in use, is positioned in the confined space 900 defined by the living body 902, and (B) while the flexible guidewire assembly 102 is movable along the confined space 900 defined by the living body 902. The first relative position is associated with the first case 201, and is the position of the source of emission of the electro-magnetic energy 202 relative to the position of the tissue features of the patient as indicated in the medical image 704 as depicted in FIG. 2 and FIG. 5. It will be appreciated that a second relative position is associated with a second case 204 as depicted, for instance, in FIG. 9.

Referring to the embodiment as depicted in FIG. 2, the medical-imaging system 702 (as depicted in FIG. 1), in use, generates a medical image 704. The medical image 704 is to be depicted to a user (a doctor, etc.) via a display device (which is known and not depicted) of the medical-imaging system 702; this is done in response to the medical-imaging system 702, in use, receiving the reflected sensor signal from the medical-imaging sensor 700 (the reflected sensor signal was reflected from the tissues of the patient toward the medical-imaging sensor 700); it will be appreciated that the sensor signal was initially emitted from (transmitted by) the medical-imaging sensor 700 (as depicted in FIG. 1) toward the tissue material of the patient.

Referring to the embodiment as depicted in FIG. 1, the method of using the flexible guidewire assembly 102 is further described below; the method includes identifying and utilizing a vein 106 positioned in the right atrium 908. The medical-imaging sensor 700 is introduced into the interior of the vein 106 so that the medical-imaging sensor 700 enters the right atrium 908. The medical-imaging system 702 establishes (makes) electrical communication with the medical-imaging sensor 700 (once the medical-imaging sensor 700 enters the right atrium 908). The medical-imaging sensor 700 is energized (activated) so that the medical-imaging sensor 700 may begin to transmit a sensor signal to the tissue of the patient (as depicted in FIG. 2), in which the tissues of the patient may reflect back at least some of the sensor signal toward the medical-imaging sensor 700 (and the medical-imaging sensor 700 may transmit the reflected sensor signal back to the medical-imaging system 702).

Referring to the embodiment as depicted in FIG. 1, the method further includes introducing the dilator assembly 103 and the energy-emitting device 104 into the vein 106 so that the dilator assembly 103 and the energy-emitting device 104 enter the right atrium 908 (as depicted in FIG. 1). The dilator assembly 103 is further moved toward the tissue wall 904 so that the dilator assembly 103 makes intimate contact with the tissue wall 904 (the doctor should be able to feel the resistance provided by the tissue wall 904 once the tip portion of the dilator assembly 103 intimately contacts the tissue wall 904) (as depicted in FIG. 1).

Referring to the embodiment as depicted in FIG. 1, the method further includes establishing electrical communication between the control assembly 800 and the energy-emitting device 104 of the flexible guidewire assembly 102 (once the flexible guidewire assembly 102 enters the right atrium 908). The energy-emitting device 104 is not yet energized to emit electromagnetic energy (that is, the control assembly 800 has not yet sent any control signal or any activation signal to the energy-emitting device 104, as depicted in FIG. 1). As a result, the position of the energy-emitting device 104 is not easily viewable or detected (detectable) in the medical image 704 generated by the medical-imaging system 702 (of FIG. 1). Once the energy-emitting device 104 is activated, the energy-emitting device 104 may be viewed or detected in the medical image 704 generated by the medical-imaging system 702 (of FIG. 1).

FIG. 3 depicts a top schematic view of the embodiment of the flexible guidewire assembly 102 and the energy-emitting device 104 of FIG. 1 (in which the energy-emitting device 104 is activated and emits a first amount of electro-magnetic energy 202, and is positioned in a spaced-apart relationship with a tissue wall 904). FIG. 4 depicts a top medical-image view of the embodiment of the flexible guidewire assembly 102 and the energy-emitting device 104 of FIG. 3 (in which the energy-emitting device 104 is activated and emits a first amount of electro-magnetic energy 202, and is positioned in a spaced-apart relationship with a tissue wall 904).

FIG. 5 depicts a top schematic view of the embodiment of the flexible guidewire assembly 102 and the energy-emitting device 104 of FIG. 1 (in which the energy-emitting device 104 is activated and emits a first amount of electro-magnetic energy 202, and is positioned proximate to (in intimate contact with) a tissue wall 904). FIG. 6 depicts a top medical-image view of the embodiment of the flexible guidewire assembly 102 and the energy-emitting device 104 of FIG. 5 (in which the energy-emitting device 104 is activated and emits the first amount of electro-magnetic energy 202, and is positioned proximate to (in intimate contact with) the tissue wall 904). Referring to the embodiment as depicted in FIG. 6, the medical-imaging system 702 (as depicted in FIG. 5), in use, generates a medical image 704 in response to the medical-imaging system 702, in use, receiving the sensor signal transmitted by the medical-imaging sensor 700 (as depicted in FIG. 5).

FIG. 3 depicts the flexible guidewire assembly 102 being moved toward the tissue wall 904, and FIG. 5 depicts the flexible guidewire assembly 102 positioned proximate to (in intimate contact with) the tissue wall 904 (all while the energy-emitting device 104 is activated and emits the first amount of electro-magnetic energy 202 without puncturing (for instance via vaporization) the tissue of the patient (such as, without causing any vaporization of the tissue of the patient).

Referring to the embodiment as depicted in FIG. 3, the method further includes activating the energy-emitting device 104 so that the position of the energy-emitting device 104 may be visualized (identified) via the medical-imaging sensor 700 and the medical-imaging system 702 of FIG. 1. The energy-emitting device 104 is configured to emit electromagnetic energy having sufficient energy intensity so that the medical-imaging sensor 700 may detect the energy-emitting device 104 (without causing any vaporization of the tissue of the patient). The energy-emitting device 104 may be selectively activated to emit different levels (at least two unique energy levels) of electromagnetic energy, such as: a first energy level (as depicted in FIG. 9) for the case where the energy-emitting device 104 is required to puncture the tissue of the patient (such as, vaporize (evaporate) a portion of the tissue of the patient), as depicted in FIG. 9. Referring back to FIG. 3, the energy-emitting device 104 may be selectively activated to emit different levels (at least two unique energy levels) of electromagnetic energy, such as: a second level (as depicted in FIG. 3) for the case where the energy-emitting device 104 is required to be detected by the medical-imaging sensor 700 but not of sufficient magnitude or strength for vaporizing (evaporating) any tissue of the patient (as depicted in FIG. 3). It will be appreciated that the amount of strength (signal strength) needed for detection (by the medical-imaging sensor 700) may be determined by experimentation and may depend on a specific make or model of the energy-emitting device 104. It will be appreciated that the amount of strength (signal strength) needed for insufficient (not strong enough) for vaporization (perforation or puncturing) of tissue may be determined by experimentation and may depend on a specific make or model of the energy-emitting device 104.

Referring to the embodiment as depicted in FIG. 3, the energy-emitting device 104 (for the second energy level, as depicted in FIG. 3) emits just enough energy so as to be visible by the medical-imaging sensor 700 (for interfering with the ICE or TEE reconstructed image) but not strong enough to puncture (perforate or vaporize) the tissue of the patient (the emitted energy is not strong enough to vaporize (evaporate or ablate) the tissue of the patient, but the emitted electromagnetic energy waves are strong enough to interfere with the ICE or TEE image to be generated by the medical-imaging system 702 (once the sensor signal is generated by and sent from the medical-imaging sensor 700 to the medical-imaging system 702). The reconstructed (generated) medial image (as depicted in FIG. 4) generated by the medical-imaging system 702 (of FIG. 1) may pinpoint (locate) the position of the energy-emitting device 104 that has become activated (that is, once the energy-emitting device 104 is activated with the desired power level for locating the position thereof without vaporizing any tissue of the patient).

Referring to the embodiment as depicted in FIG. 3, the method further includes urging the energy-emitting device 104 to emit a radio frequency signal (preferably, a radio frequency signal or energy) having a desired intensity toward the tissue of the patient. The energy-emitting device 104 is configured to provide electromagnetic energy of sufficient strength (intensity having a frequency and a power level) that may be (optimally) observed by or detected by the medical-imaging sensor 700.

Referring to the embodiment as depicted in FIG. 3, the medical-imaging sensor 700 may include an echocardiography device. The medical-imaging sensor 700 is configured to receive the signal (electromagnetic energy) that is emitted from the energy-emitting device 104. In addition, the energy-emitting device 104 is configured to transmit the electromagnetic energy to the tissue of the patient (the energy-emitting device 104 is activated by utilization of the control assembly 800).

Referring to the embodiment as depicted in FIG. 3 and FIG. 4, the flexible guidewire assembly 102 is insertable into, and is movable along, the confined space 900 defined by the living body 902. The flexible guidewire assembly 102 has the energy-emitting device 104. The energy-emitting device 104 is configured to be selectively activated in response to the energy-emitting device 104, in use, receiving a first activation signal 802 (which is transmittable from the control assembly 800); this is done in such a way that the energy-emitting device 104, once activated, emits a first amount of electro-magnetic energy 202 (as depicted in FIG. 3) under a first case 201 while the energy-emitting device 104 of the flexible guidewire assembly 102 is movable along the confined space 900 defined by the living body 902. Under the first case 201, the first amount of electro-magnetic energy 202 (as depicted in FIG. 3) has a first strength that is sufficient (strong enough) for detection by a medical-imaging sensor 700 positioned in the confined space 900 defined by the living body 902. The first strength (of the first amount of electro-magnetic energy 202) is also insufficient (not strong enough) for vaporization of any tissue of the living body 902.

Referring to the embodiment as depicted in FIG. 4, the medical-imaging system 702 (as depicted in FIG. 1), in use, generates a medical image 704 in response to the medical-imaging system 702, in use, receiving the sensor signal transmitted by the medical-imaging sensor 700 (as depicted in FIG. 3). A benefit of generating a relatively lower power electromagnetic energy signal (by the energy-emitting device 104) is that the energy-emitting device 104 may be visualized in (depicted by) the medical-imaging system 702 to the user (as depicted in FIG. 4 once the energy-emitting device 104 is activated) without causing any vaporization of the tissue of the patient; in this manner, the energy-emitting device 104 may be tracked, and displayed, by the medical-imaging system 702 (as depicted in FIG. 1). Preferably, the low power electromagnetic signal generated by the energy-emitting device 104 is sufficiently strong enough (as depicted in FIG. 4) to be distinguishable from the tissue structure (such as the anatomy of the heart of the patient) as well as other echogenic devices (such as, medical catheters, a sheath assembly, a dilator, and/or an introducer device) that are in place in the sensor field-of-view 701 (FOV) (as depicted in FIG. 3). It will be appreciated that the energy-emitting device 104 (as depicted in FIG. 3) is configured to emit sufficiently lower power in order to avoid vaporization, ablation and/or puncture of unintended tissue (the amount of sufficient power, or insufficient, may be determined by persons of skill in the art).

Referring to the embodiment as depicted in FIG. 4, the following are usages or applications for providing a more precise identification of the energy-emitting device 104 on the medical image 704 (such as, an echocardiography image, etc.). The energy-emitting device 104 may be positioned within a sheath and/or a dilator, and against the tissue wall 904 (such as, the pericardium or the pericardial sac, etc.). Once the energy-emitting device 104 is activated, the user (such as a doctor) may be able to visualize the position (spatial position) of the energy-emitting device 104 on the medical image 704 (such as, the echocardiography image), and better assess the location of the energy-emitting device 104 and/or whether access was achieved, etc.

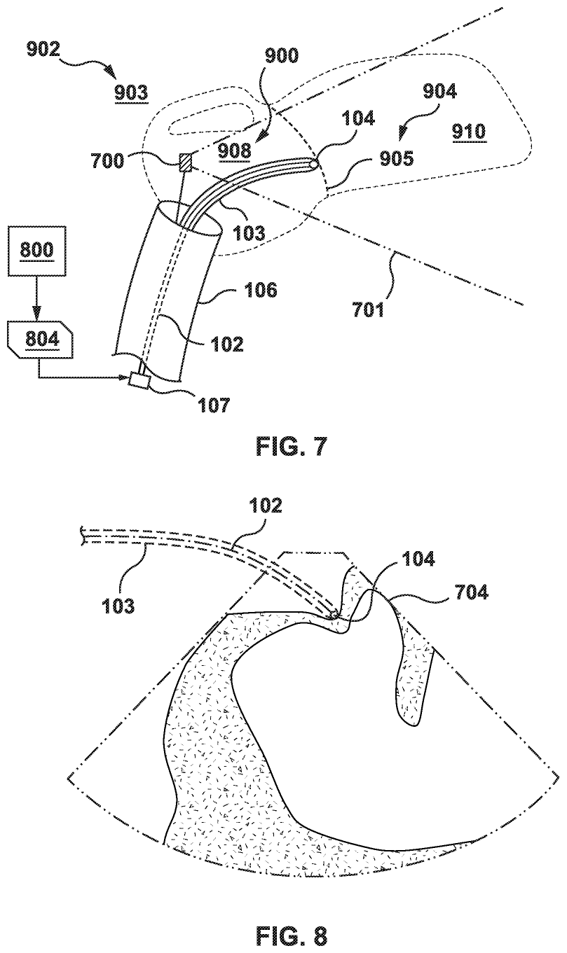

FIG. 7 depicts a top schematic view of the embodiment of the flexible guidewire assembly 102 and the energy-emitting device 104 of FIG. 1 (in which the energy-emitting device 104 is deactivated, and is positioned proximate to (in intimate contact with) a tissue wall 904). FIG. 8 depicts a top medical-image view of the embodiment of the flexible guidewire assembly 102 and the energy-emitting device 104 of FIG. 7 (in which the energy-emitting device 104 is deactivated, and is positioned proximate to (in intimate contact with) a tissue wall 904). Referring to the embodiment as depicted in FIG. 8, the medical-imaging system 702 (as depicted in FIG. 1), in use, generates the medical image 704 in response to the medical-imaging system 702, in use, receiving the sensor signal transmitted by the medical-imaging sensor 700 (as depicted in FIG. 7).

Referring to the embodiment as depicted in FIG. 7 and FIG. 8, the energy-emitting device 104 is also configured to be selectively deactivated in response to the energy-emitting device 104 receiving a first deactivation signal 804 that is transmittable from the control assembly 800; this is done in such a way that the energy-emitting device 104, in use, stops emitting the first amount of electro-magnetic energy 202. The energy-emitting 104 first stops emission of the second amount of electro-magnetic energy 206 (to ensure that the vaporization of tissue is fully stopped) before the energy-emitting 104 is reactivated for emitting another (predetermined) amount of vaporization energy (so that the energy-emitting 104 may be moved away from the place with the tissue was vaporized, etc.). For the case where the vaporization process is not automated, the doctor may be required to use personal judgement as to how long to active the vaporisation energy from the energy-emitting 104. For the case where the vaporization process is automated, the doctor may not be required to use personal judgement, and the control assembly 800 controls the duration of application of the vaporisation energy from the energy-emitting 104 (such as a predetermined energization time, etc.); moreover, if the doctor determines that perhaps more vaporization is required for application to the tissue, the control assembly 800 may receive an input indication (from the doctor) for reactivation of sufficient vaporization energy via the energy-emitting 104.

FIG. 9 depicts a top schematic view of the embodiment of the flexible guidewire assembly 102 and the energy-emitting device 104 of FIG. 1 (in which the energy-emitting device 104 is activated to emit a second amount of electro-magnetic energy 206 while positioned proximate to (in intimate contact with) a tissue wall 904). FIG. 10 depicts a top medical-image view of the embodiment of the flexible guidewire assembly 102 and the energy-emitting device 104 of FIG. 9 (in which the energy-emitting device 104 is activated to emit a second amount of electro-magnetic energy 206 while positioned proximate to (in intimate contact with) a tissue wall 904). Referring to the embodiment as depicted in FIG. 10, the medical-imaging system 702 (as depicted in FIG. 1), in use, generates a medical image 704 in response to the medical-imaging system 702, in use, receiving the sensor signal transmitted by the medical-imaging sensor 700 (as depicted in FIG. 9).

FIG. 11 depicts a top schematic view of the embodiment of the flexible guidewire assembly 102 and the energy-emitting device 104 of FIG. 1 (in which the energy-emitting device 104 is activated to emit a second amount of electro-magnetic energy 206 while positioned in a spaced-apart relationship with a tissue wall 904).

FIG. 12 depicts a top medical-image view of the embodiment of the flexible guidewire assembly 102 and the energy-emitting device 104 of FIG. 11 (in which the energy-emitting device 104 is activated to emit a second amount of electro-magnetic energy 206 while positioned in a spaced-apart relationship with a tissue wall 904). Referring to the embodiment as depicted in FIG. 12, the medical-imaging system 702 (as depicted in FIG. 1), in use, generates a medical image 704 in response to the medical-imaging system 702, in use, receiving the sensor signal transmitted by the medical-imaging sensor 700 (as depicted in FIG. 11).

Referring to the embodiment as depicted in FIG. 9 and FIG. 10, the energy-emitting device 104 is also configured to be positioned proximate to the portion of the tissue wall 904 positioned in the confined space 900 defined by the living body 902 (while the flexible guidewire assembly 102 is movable along the confined space 900 defined by the living body 902). The energy-emitting device 104 is also configured to be selectively activated in response to the energy-emitting device 104 receiving a second activation signal 806 that is transmittable from the control assembly 800; this is done in such a way that the energy-emitting device 104, in use, emits a second amount of electro-magnetic energy 206 (as depicted in FIG. 9), under a second case 204, toward the portion of the tissue wall 904 positioned in the confined space 900 defined by the living body 902 (while the energy-emitting device 104 remains positioned proximate to the portion of the tissue wall 904).

Referring to the embodiment as depicted in FIG. 9 and FIG. 10, the energy-emitting device 104 is configured to be activated to emit a level of electromagnetic energy of sufficient strength to vaporize (form) an initial pilot hole (a pilot passageway) through the tissue wall 904 (such as the atrial septum 905, as depicted in FIG. 9 and/or FIG. 10); then, the dilator assembly 103, in use, is utilized to mechanically ream out the initial pilot passageway defined through the tissue wall 904 (as depicted in FIG. 9 and/or FIG. 10) in response to the doctor (the user) physically pushing the dilator assembly 103 (after the energy-emitting device 104 has been activated to vaporize or form the initial pilot hole through the tissue wall).

Referring to the embodiment as depicted in FIG. 9 and FIG. 10, under the second case 204, the second amount of electro-magnetic energy 206 (while the energy-emitting device 104 remains positioned proximate to a portion of the tissue wall 904) has a second strength (that is, the second amount of electro-magnetic energy 206) that may be sufficient for detection by the medical-imaging sensor 700 (as depicted in FIG. 1) while the medical-imaging sensor 700 is positioned for detecting the second amount of electro-magnetic energy 206 emanating from the energy-emitting device 104. The second strength (of the second amount of electro-magnetic energy 206) is also sufficient for vaporization of the portion of the tissue wall 904; this is done in such a way that the second amount of electro-magnetic energy 206, in use, forms a passageway 906 (an initial pilot hole) extending through the tissue wall 904. It will be appreciated that the amount of strength (signal strength) needed for (sufficient for) vaporization of tissue may be determined by experimentation and may depend on a specific make or model of the energy-emitting device 104.

FIG. 13 depicts a top schematic view of the embodiment of the flexible guidewire assembly 102 and the energy-emitting device 104 of FIG. 1 (in which the energy-emitting device 104 is deactivated while positioned in a spaced-apart relationship with a tissue wall 904). FIG. 14 depicts a top medical-image view of the embodiment of the flexible guidewire assembly 102 and the energy-emitting device 104 of FIG. 13 (in which the energy-emitting device 104 is deactivated while positioned in a spaced-apart relationship with a tissue wall 904). Referring to the embodiment as depicted in FIG. 14, the medical-imaging system 702 (as depicted in FIG. 1), in use, generates a medical image 704 in response to the medical-imaging system 702, in use, receiving the sensor signal transmitted by the medical-imaging sensor 700 (as depicted in FIG. 13).

Referring to the embodiment as depicted in FIG. 13 and FIG. 14, the energy-emitting device 104 is also configured to be selectively deactivated in response to the energy-emitting device 104 receiving a second deactivation signal 808 that is transmittable from the control assembly 800; this is done in such a way that the energy-emitting device 104, in use, stops emitting the second amount of electro-magnetic energy 206.

Referring to the embodiment as depicted in FIG. 13 and FIG. 3, the energy-emitting device 104 is also configured to be selectively activated in response to the energy-emitting device 104 (in use) receiving the first activation signal 802 (as depicted in FIG. 3, for instance) that is transmittable from the control assembly 800 once the energy-emitting device 104, in use, stops emission of the second amount of electro-magnetic energy 206; this is done in such a way that the energy-emitting device 104, in use, emits the first amount of electro-magnetic energy 202 under the first case 201.

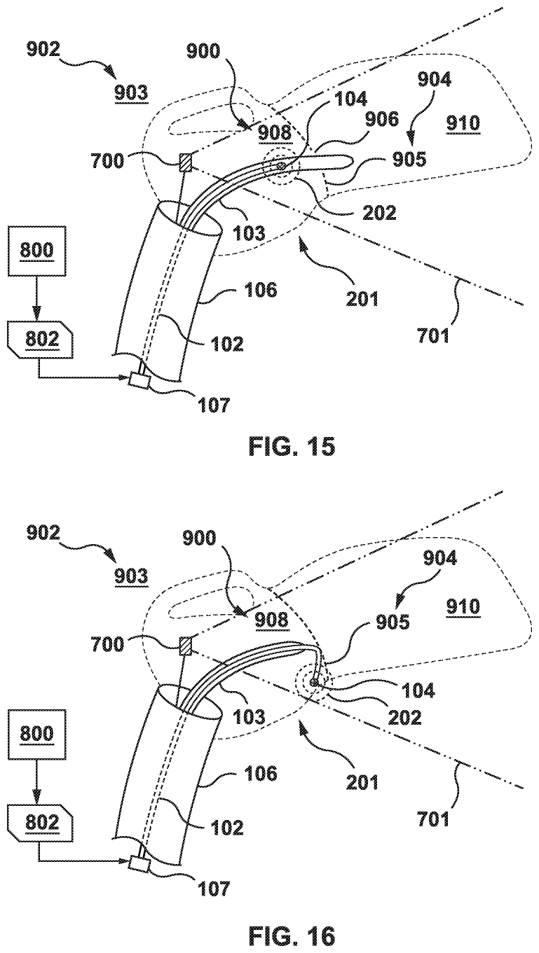

FIG. 15 and FIG. 16 depict top schematic views of the embodiments of the flexible guidewire assembly 102 and the energy-emitting device 104 of FIG. 1 (which depict cases to be, preferably, avoided).

Referring to the embodiment as depicted in FIG. 15, the tip of the dilator assembly 103 is pushed (inadvertently) with enough force (too much force) to (inadvertently) form the passageway 906 through the tissue wall 904 while the energy-emitting device 104 emits the first amount of electro-magnetic energy 202. For this case, the energy-emitting device 104 was not utilized for the formation of the passageway 906. For this case, the energy-emitting device 104 may be deactivated or may be withdrawn and deactivated. For this case, it may be preferable not to activate the energy-emitting device 104 so that the energy-emitting device 104 may then emit the second amount of electro-magnetic energy 206 (as depicted in FIG. 9).

Referring to the embodiment as depicted in FIG. 16, there is depicted a case in which the energy-emitting device 104 and the flexible guidewire assembly 102 were pushed toward the tissue wall 904, and the flexible guidewire assembly 102 became bent and was deflected by the tissue wall 904, and then the flexible guidewire assembly 102 travels (moves) away from the tissue wall 904. For this case, the energy-emitting device 104 was not placed proximate to the tissue wall 904, and, therefore, should not be utilized (activated) for the formation of the passageway 906. For this case, the energy-emitting device 104 may be deactivated and/or may be withdrawn and deactivated. For this case, it would be preferable not to activate the energy-emitting device 104 so that the energy-emitting device 104 does not emit the second amount of electro-magnetic energy 206 (as depicted in FIG. 9) and thereby avoid any inadvertent damage to surrounding tissue of the patient.

The following is offered as further description of the embodiments, in which any one or more of any technical feature (described in the detailed description, the summary and the claims) may be combinable with any other one or more of any technical feature (described in the detailed description, the summary and the claims). It is understood that each claim in the claims section is an open ended claim unless stated otherwise. Unless otherwise specified, relational terms used in these specifications should be construed to include certain tolerances that the person skilled in the art would recognize as providing equivalent functionality. By way of example, the term perpendicular is not necessarily limited to 90.0 degrees, and may include a variation thereof that the person skilled in the art would recognize as providing equivalent functionality for the purposes described for the relevant member or element. Terms such as “about” and “substantially”, in the context of configuration, relate generally to disposition, location, or configuration that are either exact or sufficiently close to the location, disposition, or configuration of the relevant element to preserve operability of the element within the invention which does not materially modify the invention. Similarly, unless specifically made clear from its context, numerical values should be construed to include certain tolerances that the person skilled in the art would recognize as having negligible importance as they do not materially change the operability of the invention. It will be appreciated that the description and/or drawings identify and describe embodiments of the apparatus (either explicitly or inherently). The apparatus may include any suitable combination and/or permutation of the technical features as identified in the detailed description, as may be required and/or desired to suit a particular technical purpose and/or technical function. It will be appreciated that, where possible and suitable, any one or more of the technical features of the apparatus may be combined with any other one or more of the technical features of the apparatus (in any combination and/or permutation). It will be appreciated that persons skilled in the art would know that the technical features of each embodiment may be deployed (where possible) in other embodiments even if not expressly stated as such above. It will be appreciated that persons skilled in the art would know that other options would be possible for the configuration of the components of the apparatus to adjust to manufacturing requirements and still remain within the scope as described in at least one or more of the claims. This written description provides embodiments, including the best mode, and also enables the person skilled in the art to make and use the embodiments. The patentable scope may be defined by the claims. The written description and/or drawings may help to understand the scope of the claims. It is believed that all the crucial aspects of the disclosed subject matter have been provided in this document. It is understood, for this document, that the word “includes” is equivalent to the word “comprising” in that both words are used to signify an open-ended listing of assemblies, components, parts, etc. The term “comprising”, which is synonymous with the terms “including,” “containing,” or “characterized by,” is inclusive or open-ended and does not exclude additional, unrecited elements or method steps. Comprising (comprised of) is an “open” phrase and allows coverage of technologies that employ additional, unrecited elements. When used in a claim, the word “comprising” is the transitory verb (transitional term) that separates the preamble of the claim from the technical features of the invention. The foregoing has outlined the non-limiting embodiments (examples). The description is made for particular non-limiting embodiments (examples). It is understood that the non-limiting embodiments are merely illustrative as examples.

What is claimed is:

1. An apparatus, comprising:
a flexible guidewire assembly being insertable into, and being movable along, a confined space defined by a living body; and
the flexible guidewire assembly having an energy-emitting device; and
the energy-emitting device being configured to be selectively activated in response to the energy-emitting device, in use, receiving a first activation signal being transmittable from a control assembly in such a way that the energy-emitting device, once activated, emits a first amount of electro-magnetic energy under a first case while the energy-emitting device of the flexible guidewire assembly is movable along the confined space defined by the living body; and
wherein under the first case, the first amount of electro-magnetic energy has a first strength that is:
sufficient for detection by a medical-imaging sensor positioned in the confined space defined by the living body; and
insufficient for vaporization of any tissue of the living body.

2. The apparatus of claim 1, wherein:
the energy-emitting device is also configured to be selectively deactivated in response to the energy-emitting device receiving a first deactivation signal being transmittable from the control assembly in such a way that the energy-emitting device, in use, stops emitting the first amount of electro-magnetic energy.

3. The apparatus of claim 1, wherein:
the medical-imaging sensor is configured to be in signal communication with a medical-imaging system while the medical-imaging sensor, in use, is positioned in the confined space defined by the living body; and
the medical-imaging system is configured to receive a sensor signal transmitted by the medical-imaging sensor while the medical-imaging sensor, in use, is positioned in the confined space defined by the living body; and
the medical-imaging system is configured to display, to a user, a medical image depicting a first relative position of an emission of the first amount of electro-magnetic energy in response to the medical-imaging system, in use, receiving the sensor signal transmitted by the medical-imaging sensor while the medical-imaging sensor, in use, is positioned in the confined space defined by the living body, and while the flexible guidewire assembly is movable along the confined space defined by the living body.

4. The apparatus of claim 3, wherein:
the energy-emitting device is also configured to be positioned proximate to a portion of a tissue wall positioned in the confined space defined by the living body while the flexible guidewire assembly is movable along the confined space defined by the living body; and
the energy-emitting device is also configured to be selectively activated in response to the energy-emitting device receiving a second activation signal being transmittable from the control assembly in such a way that the energy-emitting device, in use, emits a second amount of electro-magnetic energy, under a second case, toward the portion of the tissue wall positioned in the confined space defined by the living body while the energy-emitting device remains positioned proximate to the portion of the tissue wall.

5. The apparatus of claim 4, wherein:
under the second case, the second amount of electro-magnetic energy, while the energy-emitting device remains positioned proximate to the portion of the tissue wall, has a second strength that is:
sufficient for detection by the medical-imaging sensor while the medical-imaging sensor is positioned for detecting the second amount of electro-magnetic energy emanating from the energy-emitting device; and
sufficient for vaporization of the portion of the tissue wall in such a way that the second amount of electro-magnetic energy, in use, forms a passageway extending through the tissue wall.

6. The apparatus of claim 5, wherein:
the energy-emitting device is also configured to be selectively deactivated in response to the energy-emitting device receiving a second deactivation signal being transmittable from the control assembly in such a way that the energy-emitting device, in use, stops emitting the second amount of electro-magnetic energy.

7. The apparatus of claim 6, wherein:
the energy-emitting device is also configured to be selectively activated in response to the energy-emitting device, in use, receiving the first activation signal being transmittable from the control assembly once the energy-emitting device, in use, stops emission of the second amount of electro-magnetic energy in such a way that the energy-emitting device, in use, emits the first amount of electro-magnetic energy under the first case.

8. An apparatus, comprising:
a control assembly being configured to transmit a first activation signal to an energy-emitting device of a flexible guidewire assembly being configured to be selectively activated in response to the energy-emitting device, in use, receiving the first activation signal in such a way that the energy-emitting device, once activated, emits a first amount of electro-magnetic energy under a first case while the energy-emitting device of the flexible guidewire assembly is movable along a confined space defined by a living body, in which the flexible guidewire assembly is insertable into, and is movable along, a confined space defined by a living body; and
wherein under the first case, the first amount of electro-magnetic energy has a first strength that is:
sufficient for detection by a medical-imaging sensor positioned in the confined space defined by the living body; and
insufficient for vaporization of any tissue of the living body.

9. The apparatus of claim 8, wherein:
the control assembly is also configured to transmit a first deactivation signal to the energy-emitting device to selectively deactivate the energy-emitting device in such a way that the energy-emitting device, in use, stops emitting the first amount of electro-magnetic energy.

10. The apparatus of claim 8, wherein:
the control assembly is also configured to transmit a second activation signal to the energy-emitting device to selectively activate the energy-emitting device in such a way that the energy-emitting device, in use, emits a second amount of electro-magnetic energy, under a second case, toward a portion of a tissue wall positioned in the confined space defined by the living body while the energy-emitting device remains positioned proximate to the portion of the tissue wall.

11. The apparatus of claim 10, wherein:
under the second case, the second amount of electro-magnetic energy, while the energy-emitting device remains positioned proximate to the portion of the tissue wall, has a second strength that is:
sufficient for detection by the medical-imaging sensor while the medical-imaging sensor is positioned for detecting the second amount of electro-magnetic energy emanating from the energy-emitting device; and
sufficient for vaporization of the portion of the tissue wall in such a way that the second amount of electro-magnetic energy, in use, forms a passageway extending through the tissue wall.

12. The apparatus of claim 10, wherein:
the control assembly is also configured to transmit a second deactivation signal to the energy-emitting device to selectively deactivate the energy-emitting device in such a way that the energy-emitting device, in use, stops emitting the second amount of electro-magnetic energy.

13. A method of utilizing a flexible guidewire assembly including an energy-emitting device, the method comprising:
identifying and utilizing a vein positioned in a right atrium of a patient; and
introducing a medical-imaging sensor into an interior of the vein so that the medical-imaging sensor enters the right atrium; and
establishing electrical communication between a medical-imaging sensor and a medical-imaging system; and
energizing the medical-imaging sensor so that the medical-imaging sensor transmits a sensor signal to the tissue of the patient, in which the tissue of the patient reflect back at least some of the sensor signal toward the medical-imaging sensor, and the medical-imaging sensor, in use, transmits a reflected sensor signal back to the medical-imaging system; and
introducing a dilator assembly and the energy-emitting device of the flexible guidewire assembly into the interior of the vein so that the dilator assembly and the energy-emitting device enter the right atrium; and
moving the dilator assembly toward a tissue wall so that the dilator assembly makes intimate contact with the tissue wall; and
establishing electrical communication between a control assembly and the energy-emitting device of the flexible guidewire assembly.

14. The method of claim 13, further comprising:
activating the energy-emitting device to emit a first amount of electro-magnetic energy once the energy-emitting device is positioned in a spaced-apart relationship with a tissue wall so that a position of the energy-emitting device is visualized via synergistic cooperation of the medical-imaging sensor and the medical-imaging system without causing any vaporization of the tissue of the patient.

15. The method of claim 14, further comprising:
activating the energy-emitting device to emit a second amount of electro-magnetic energy once the energy-emitting device is positioned proximate to the tissue wall so that the energy-emitting device forms a passageway through the tissue wall by vaporizing a portion of the tissue wall.
